(19) United States
(12) Patent Application Publication
(10) Pub. No.: US 2015/0005657 A1
(43) Pub. Date: Jan. 1, 2015

(54) GAS SAMPLING DEVICE AND METHOD

(71) Applicant: KONINKLIJKE PHILIPS N.V., EINDHOVEN (NL)

(72) Inventors: Tamara Mathea Elisabeth Nijsen, Weert (NL); Anthonius Petrus Gerardus Emanuel Janssen, Berghem (NL)

(73) Assignee: Koninklijke Philips N.V., Eindhoven (NL)

(21) Appl. No.: 14/377,995

(22) PCT Filed: Feb. 8, 2013
(86) PCT No.: PCT/IB2013/051064

§ 371 Date: Aug. 11, 2014

Related U.S. Application Data
(60) Provisional application No. 61/596,781, filed on Feb. 9, 2012.

(51) Int. Cl. A61B 5/097; A61M 16/08; A61B 5/08

(52) U.S. Cl. CPC A61B 5/097; A61B 5/082; A61M 16/085
USPC 600/532; 600/543

(57) ABSTRACT

The invention relates to a gas sampling device (100) for collecting gas samples from a patient or other gas sources such as industrial processes. The gas sampling device has a flow controller (114) and a flow sensor (112) for controlling a gasflow from the gas source created by a gas pump (113). The gas flow is pulled through an associated absorbent tube (180) located up-stream relative to the flow sensor and pump.

### FIELD OF THE INVENTION

The invention relates to a gas sampling device, particularly to a breathing gas sampling device for medical use.

### BACKGROUND OF THE INVENTION

Current practice for taking breath samples is to let the patient breath in a bag for a while. Afterwards the collected breath is pushed or pulled through a sorbent tube for collecting Volatile Organic Compounds from the breath sample.

The pushing or pulling of the breath sample may be performed by a pump. However, current available pumps are very bulky and setting them up together with tubing fit to the sorbent tubes and the breathing bag takes time and is not suitable to apply at a patient's bedside.

Furthermore it may be a problem to collect breathing samples from patients which are not able to breathe in a bag e.g. because the patients are being mechanically ventilated.

U.S. Pat. No. 5,826,577 discloses a breath gas analysis module for supplying a sample gas to a gas detector which includes a manifold having a body defining a chamber therein. The chamber includes a main passage extending through the manifold body, an outlet passage extending into the manifold body from an outlet end at a base of the manifold body and toward the main passage and a metering orifice extending between and in fluid communication with the main passage and an interior end of the outlet passage. An inlet tube is attached to the manifold body at one end of the main passage and defines an internal passage in fluid communication with the main passage. A collection tube is attached to the manifold body at another end of the main passage and defines an internal passage in fluid communication with the main passage. A check valve is positioned within the inlet tube internal passage and is oriented to permit fluid flow into, but not back from, the main passage. A resilient gas reservoir is attached to a free end of the collection tube opposite an end thereof attached to the manifold body. The resilient gas reservoir defines a storage chamber therein which is in fluid communication with the collection tube internal passage.

Due to the above problems the inventor of the present invention has appreciated that an improved gas sampling device is of benefit, and has in consequence devised the present invention.

### SUMMARY OF THE INVENTION

It would be advantageous to achieve improvements of gas sampling devices. In general, the invention preferably seeks to alleviate or eliminate one or more of the above mentioned disadvantages singly or in any combination. In particular, it may be seen as an object of the present invention to provide a method that solves the above mentioned problems of unsuitableness of known devices to be used at a patient's bedside and/or to be used with patients that not able the breathe themselves, or other problems, of the prior art.

To better address one or more of these concerns, in a first aspect of the invention a gas sampling device is presented that comprises
an inlet for receiving gas,
a pump fluidly connected to the inlet for drawing the gas through the inlet,
a flow sensor for measuring the flow of gas drawn through the inlet,
a flow controller for controlling the pump in dependence of the measured flow of the gas.

The pump advantageously enables collection of gas samples from gas sources which does not provide sufficient pressure to create a flow into the gas sampling device.

Thus, for medical applications the pump enables collection of breathing gas from patients which are not able to actively blow breathing air into a bag or a gas sampling device.

In an embodiment the gas sampling device is configured to enable connection with an associated gas collector in a way which enables the gas drawn by the pump to flow through the gas collector so as to enable the gas collector to extract content of the gas. For example, the gas collector may be a sorbent tube which is capable of extracting volatile organic compounds from the gas or breathing gas. The gas collector may be housed by some part of the gas sampling device at a location after the inlet, or the inlet of the gas sampling device may be configured be to enable connection with the associated gas collector so that gas is drawn through the gas collector and into the inlet.

In an embodiment the pump and the flow sensor are located downstream relative to the gas collector. By such a downstream location it may be avoided that the gas content which is extracted by the gas collector is contaminated by the pump and the flow sensor.

In an embodiment the flow controller is capable of controlling the pump so as to draw a preset flow amount of gas via the inlet, such as a preset mass or volume of the gas flow. In another embodiment the flow controller is capable of controlling the pump so as to draw breathing gas via the inlet with a preset flow. The capability of controlling the flow amount and/or the flow of gas which passes through the gas collector may be important for ensuring consistent and reliable analysis results. Also, different samples of gas can only be compared if the gas content is extracted from the gas flows and gas flow amounts which do not vary between the different samples. In an embodiment the preset flow amount and/or the preset flow may be adjustable and set-able via a user input.

In an embodiment the pump and the flow controller is powered by a battery comprised by the gas sampling device. The use of a battery powered gas sampling device may make the device more portable which may be particularly advantageous for medical use.

In an embodiment the inlet of the gas sampling device is connectable with medical equipment such as a sample bag for storing breathing gas samples or a mechanical ventilator for automatically ventilating a patient.

A second aspect of the invention relates to a gas sampling assembly which comprises:
a gas sampling device according to the first aspect, and
a gas collector fluidly connectable with the gas sampling device.

In an embodiment the gas collector comprises a connector which is connectable with medical equipment such as a sample bag or mechanical ventilator.

In a third aspect the invention relates to method for analysing gas, the method comprises:
providing a gas sampling device according to the first aspect,
connecting a gas collector to the gas sampling device,
connecting an inlet of the gas collector or the inlet of the gas sampling device to a source containing the gas to be analyzed, and
activating the gas sampling device for pulling gas through the gas collector.

In summary the invention relates to a gas sampling device for collecting gas samples from a patient or other gas sources such as industrial processes. The gas sampling device has a flow controller and a flow sensor for controlling a gas flow from the gas source which is created by a gas pump. The gas flow is pulled through an associated sorbent tube, i.e. a gas collector, preferably located upstream relative to the flow sensor and pump.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows a gas sampling assembly 101 comprising a gas sampling device 100, and

FIG. 2 illustrates a method of an embodiment of the invention.

### DETAILED DESCRIPTION OF THE EMBODIMENTS

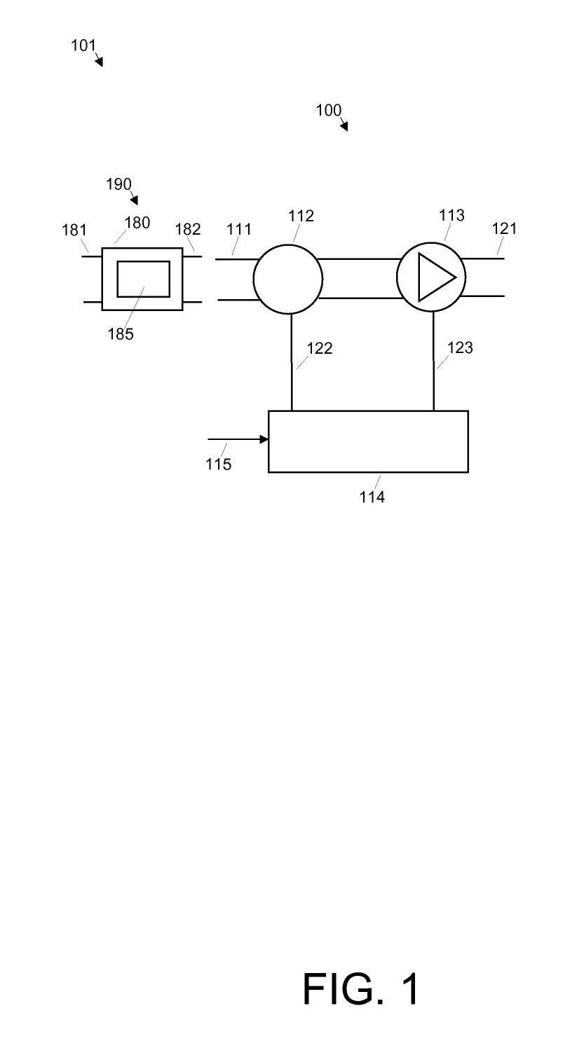

FIG. 1 shows a gas sampling assembly 101 comprising a gas sampling device 100 and a gas collector 190 such as a sorbent compartment 180 fluidly connectable with the gas sampling device 100.

The gas sampling device is configured with an inlet 111 for receiving gas, a pump 113 fluidly connected with the inlet 111 for drawing a flow of the gas through the inlet, a flow sensor 112 located for measuring the flow of gas drawn through the inlet 111 by the pump 113, and an outlet 121 for expelling the gas drawn by the pump.

Both the pump 113 and the flow sensor 112 are in fluid communication with each other and the inlet 111. The pump 113 and the flow sensor 112 are located downstream relative to the inlet 111 so that gas is pulled via the inlet 111 through the pump and the flow sensor. The flow sensor 112 may be located upstream relative to the pump 113 so that the flow sensor 112 is located between the inlet 111 and the pump 113 as shown in FIG. 1, or the flow sensor 112 may be located downstream relative to the pump 113 (not shown), i.e. after the pump.

By locating the flow sensor 112 upstreamstream relative to the pump, the gas flow is pulled through the flow sensor 112 and, thereby, the flow sensor may generate more accurate measurements of the flow and flow amount of gas pulled through the associated sorbent compartment as compared to a flow sensor 112 located downstream relative to the pump 113.

The gas sampling device 100 further comprises a flow controller 114 for controlling the pump 113 in dependence of the measured flow of the gas. For example, the controller 114 may be configured to control the pump in dependence of measured flow values so as to draw a preset flow amount such as a mass or volume of gas from the inlet, and/or so as to draw breathing gas via the inlet with a preset flow. Thus, the controller receives measured flow values from the flow sensor 112 via a connection 122 (preferably a wired connection), and the controller provides a control signal or a drive signal to the pump 113 via a connection 123 (preferably a wired connection).

The controller 114 may have a user input function 115 in the form of a keyboard, a touch sensitive screen or similar user input means. In embodiments the flow amount and/or the flow to be controlled by the controller 114 are set-able via the user input 115.

The flow sensor 112 may be a mass flow sensor which measures the mass of gas passing through the sensor per time unit or a volumetric flow sensor which measures the volume of gas which passes the sensor per time unit.

The pump 113 may be a vacuum pump such as a diaphragm pump. Other types of air pumps particularly for medical usage comprising peristaltic pumps and plunger pumps could be used. However these pumps cannot create a vacuum and therefore they cannot be placed downstream relative to gas collector 190 but should be placed upstream relative to the gas collector 190 so as to pump gas into the gas collector 190 by creating a pressure. The upstream location may imply that the gas content which is selected by the downstream gas collector becomes contaminated.

The gas sampling device 100 may be configured for sampling breathing gas in a medical environment. For example, breathing gas may be collected from a breathing bag. Since the gas sampling device has a vacuum pump, it is possible to collect breathing gas samples from mechanically ventilated patients which are not able to breathe in a bag. For example, a breathing sample may be collected from a side stream of a mechanical ventilator.

The content of the gas samples may be analyzed by means of gas collector 190 which is able to extract contents of the gas flowing through the gas collector 190. For example, the gas collector 190 may be a sorbent tube which is able to sorbe and store different contents of the gas which flows through the sorbent tube. The stored gas content in the sorbent tube can be analyzed by sorbent tube analysing apparatuses. The gas collector 190 could also be cold-trap device which condenses gas and gas content into a liquid or solid which can be analyzed subsequently or the gas collector may be a gas analysing device capable of analysing the gas or gas content real-time without use of other analysing devices.

The gas sampling device 100 is configured so that an associated gas collector 190 can be connected to the sampling device or accommodated by the gas sampling device in a way which enables the gas drawn by the pump to flow though through the gas collector 190 or sorbent tube 180 so as to enable the gas collector to extract content of the gas. Preferably, the sorbent tube 180 or other gas collector 190 should be located upstream relative to the flow sensor 112 and the pump 113 to avoid contamination of the gas which is sorbed by the sorbent tube.

For example, the inlet 111 of the gas sampling device 100 may be configured to enable an airtight connection with an associated gas collector 190 such as a sorbent compartment 180 which is configured for containing sorbent material 185 for sorbing content of the gas drawn through the inlet. For that purpose, the gas collector 190 such as the sorbent compartment 180 has an output connector 182 which is connectable with the inlet 111, and an input connector 181 which enables connection of the gas collector 190 to the gas to be analyzed. For example, the input connector 181 may be connectable with medical equipment such as breathing bags or mechanical patient ventilators, e.g. via a side stream tube of a mechanical ventilator. The sorbent compartment 180 may be a sorbent tube itself which is provided with some encapsulation 180 and an inlet 181 and an outlet 182.

Alternatively, the gas sampling device 100 may be configured with some chamber, preferably located upstream relative to the pump and the flow sensor and downstream relative to the inlet 111, which chamber is configured to accommodate a gas collector 190, a sorbent compartment 180 or sorbent material 185 so that gas flows via the inlet 111 though the accommodated gas collector, sorbent compartment or sorbent material. When the gas collector 190 is not connected to the gas sampling device via the inlet 111 but is otherwise accommodated by the gas sampling device, the inlet 111 may be configured to be connectable with medical equipment such as breathing bags or mechanical patient ventilators.

The pump 112 and the flow controller 113 of the gas sampling device 100 may be powered by a battery comprised by the gas sampling device. Thereby, a gas sampling device for medical use can easily be used in different environments of a hospital and inconvenient power cables are avoided.

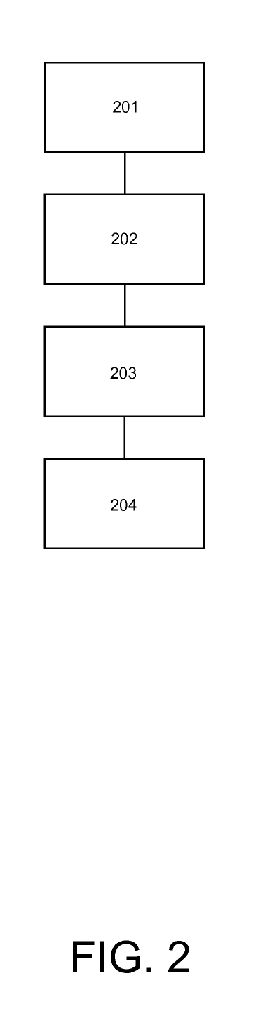

FIG. 2 illustrates steps of a method according to an aspect of the invention, where
Step 201 comprises providing the gas sampling device 100,
Step 202 comprises connecting a gas collector 190 or sorbent compartment 180 containing a sorbent tube 185 to the inlet 111 of the gas sampling device,
Step 203 connecting an inlet 181 of the gas collector 190 or the sorbent compartment 180 to a source containing the gas to be analyzed, and
Step 204 comprises activating the gas sampling device for pulling gas through the sorbent tube 185 or the gas collector 190.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single controller or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. A gas sampling device comprising
an inlet for receiving gas,
a pumps fluidly connected to the inlet for drawing the gas through the inlet,
a flow sensor for measuring the flow of gas drawn through the inlet,
a flow controller for controlling the pump in dependence of the measured flow of the gas,
wherein the flow controller is capable of controlling the pump so as to draw a preset flow amount of gas via the inlet.

2. A gas sampling device according to claim 1, for sampling breathing gas.

3. A gas sampling device according to claim 1, which is configured to enable connection with an associated gas collector in a way which enables the gas drawn by the pump to flow through the gas collector so as to enable the gas collector to extract content of the gas.

4. A gas sampling device according to claim 3, where the inlet is configured to enable the connection with the associated gas collector so that gas is drawn through the gas collector and into the inlet.

5. A breathing gas sampling device according to claim 1, where the pump and the flow sensor are located downstream relative to the gas collector.

6. (canceled)

7. (canceled)

8. A breathing gas sampling device according to claim 1, where the pump and the flow controller is powered by a battery comprised by the gas sampling device.

9. A breathing gas sampling device according to claim 1, where the inlet is connectable with medical equipment.

10. A gas sampling assembly comprising
a gas sampling device according to claim 1, and
a gas collector fluidly connectable with the gas sampling device.

11. A gas sampling assembly according to claim 10, where the gas collector comprises a connector which is connectable with medical equipment.

12. A method for analysing gas, the method comprises
providing a gas sampling device according to claim 1,
connecting a gas collector to the gas sampling device,
connecting an inlet of the gas collector or the inlet of the gas sampling device to a source containing the gas to be analyzed, and
activating the gas sampling device for pulling gas through the gas collector so as to draw a preset flow amount of gas.
